(19) United States
(12) Patent Application Publication
(10) Pub. No.: US 2016/0271301 A1
(43) Pub. Date: Sep. 22, 2016

(54) Hybrid Corrosion Inhibiting and Bio-Functional Coatings for Magnesium-Based Materials for Development of Biodegradable Metallic Implants

(71) Applicants: Siddarth SENTHIL-KUMAR, Lincoln, NE (US); WICHITA STATE UNIVERSITY, Wichita, KS (US)

(72) Inventors: Anil Mahapatro, Wichita, KS (US); Siddarth Senthil-Kumar, Lincoln, NE (US)

(21) Appl. No.: 15/035,076

(22) PCT Filed: Nov. 6, 2014
(86) PCT No.: PCT/US14/64377

§ 371 Date: May 6, 2016

Related U.S. Application Data
(60) Provisional application No. 61/901,764, filed on Nov. 8, 2013.

(51) Int. Cl. A61L 31/08; A61L 31/14; A61L 31/16; A61L 27/04; A61L 27/30; A61L 27/58; A61L 27/54; C25D 7/00; C25D 3/20; C25D 3/40; C25D 5/42; C23C 28/02; C23C 18/16; C23C 18/31; C23C 18/52; A61L 31/02

(52) U.S. Cl. CPC A61L 31/088; A61L 31/022; A61L 31/148; A61L 31/16; A61L 27/047; A61L 27/306; A61L 27/58; A61L 27/54; C25D 7/00; C25D 3/20; C25D 3/40; C25D 5/42; C23C 28/025; C23C 18/1653; C23C 18/31; C23C 18/52; A61L 2400/18

(57) ABSTRACT

An implantable device and methods of inhibiting corrosion on a magnesium-based implantable device are described. The implantable device comprising a body having an external surface. The body or one or more structural members thereof is formed at least in part by metallic magnesium or an alloy thereof. The implantable device comprises a biocorrosion-inhibiting film on at least a portion of the external surface, wherein the biocorrosion-inhibiting film comprises a metal.

### CROSS-REFERENCE TO RELATED APPLICATIONS

The present application claims the priority benefit of U.S. Provisional Patent Application Ser. No. 61/901,764, filed Nov. 8, 2013, entitled HYBRID CORROSION INHIBITING AND BIO-FUNCTIONAL COATINGS FOR MAGNESIUM BASED MATERIALS FOR DEVELOPMENT OF BIODEGRADABLE METALLIC IMPLANTS, incorporated by reference in its entirety herein.

### BACKGROUND OF THE INVENTION

1. Field of the Invention

The present invention relates to corrosion-resistant magnesium-based medical implants and methods of making the same.

2. Description of Related Art

A medical implant is a device used to replace/repair a missing/damaged biological structure. Bone implants, arterial stents, and ear implants etc. are some of the common types of implants used. A medical implant has two functional requirements biocompatibility and functional performance. The implant when placed in the human body must not elicit a toxic response on its interaction with the tissues. When placed in the human body it must not degrade (permanent implants) within the body. An implant placed in the human body must provide adequate mechanical strength and stiffness which depends on the type of application. Some common biomaterials are metals, polymers, composites and ceramics. Some implants, like bone screws, plates and arterial stents have no clinical rationale for their permanent presence thus necessitating their removal after a given period of time. In order to prevent another surgery biodegradable implants have been developed. Biodegradable implants degrade over a period of time. The major idea of biodegradable implant is to dissolve after the specified functional period with no harmful effects to the human body. The rate of degradation needs to match the tissue healing rate. Biodegradable implants can be made of polymers, ceramics, and metals.

Polymers have been used in medicine extensively, e.g., as catheters, syringes, tissue regeneration and cell encapsulation. Based on their backbone reactivity they can be broadly classified into degradable and non-degradable. Biodegradable polymers can be used for drug delivery and scaffolding applications but not for skeletal reconstruction owing to its poor strength in comparison with metals

Metals have also been extensively used for various types of implants. Some advantages of metal implants are better wear resistance, high loading capacity and excellent toughness. Some metals commonly used are 18-8 stainless steel, 316-L stainless steel, cobalt chromium, and Ti, iron, zinc, magnesium, and alloys. The current trend for bio metallic implants is biodegradable metals. In order to prevent a second surgery and maintain the same compressive strength similar to permanent implants biodegradable metallic implants are being researched. Examples of biodegradable metals include iron, magnesium, and zinc. Iron has a slow degradation rate in the body, which can be increased by alloying with less nobler metals to induce intergranular corrosion. Degradation rate of magnesium is very high, magnesium is found to degrade in 30 days in human body conditions. The degradation rate can be slowed down by selective alloying or surface coating. Alloying magnesium with nobler metals increases corrosion resistance.

Ceramics are commonly used in bone and dental applications. Some common types of ceramics are alumina, zirconia, and calcium phosphate. Most of these ceramics are bio-conductive. They are biocompatible and they permit growth of cells on their surface. Most ceramic implants are used in bone screws, bone spacers, bone joints, bone plates, and artificial teeth for dental applications. Hydroxyapatite is another type of bio-inductive implant. Hydroxy apatite is a calcium phosphate ceramic which has chemical and structural properties similar to bone minerals. Nanostructured hydroxyapatite is found to have better bioactivity compared to coarse ones. Most modern hip replacement implants are coated with hydroxy apatite to promote cell growth.

Magnesium continues to be a desired material for biodegradable implants and research has been aimed at reducing corrosion of such implants. There is still a need for improved biodegradable implant materials and strategies for making the same.

### SUMMARY OF THE INVENTION

The present invention is broadly concerned with an implantable device comprising a body having an external surface. The body or one or more structural members thereof is formed at least in part by metallic magnesium or an alloy thereof. Advantageously, the implantable device comprises a biocorrosion-inhibiting film on at least a portion of the external surface, wherein the biocorrosion-inhibiting film comprises a metal.

Methods of inhibiting corrosion on a magnesium-based implantable device are also described herein. The methods generally comprise providing a body having an external surface. The body or one or more structural members thereof is formed at least in part by metallic magnesium or an alloy thereof. A biocorrosion-inhibiting film is deposited on at least a portion of the external surface, wherein the biocorrosion-inhibiting film comprises a metal.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flow chart of the steps for electroplating iron on AZ31 magnesium alloy with an intermediate copper layer;

FIG. 2 shows the mass loss analysis images of the ionic liquid test conducted;

FIG. 3 is an SEM image of iron-coated AZ31 Magnesium without an intermediate sublayer;

FIG. 4 is a photographic image of zinc immersion coated AZ31 magnesium alloy;

FIG. 5 is a photographic image of copper coated AZ31 magnesium alloy;

FIG. 6 is a photographic image of iron coated AZ31 magnesium alloy with an intermediate copper layer;

FIG. 7 is an optical microscope image of zinc immersion coated AZ31 magnesium alloy at a magnification of A)10× and B) 20×X;

FIG. 8 is an optical microscope image of copper electroplated AZ31 magnesium alloy at a magnification of A)10× and B) 20×X;

FIG. 9 is an optical microscope image of iron electroplated AZ31 magnesium alloy with an intermediate copper layer at a magnification of A)10× and B) 20×;

FIG. 10 is an SEM image of iron coated AZ31 magnesium alloy without an intermediate sublayer at magnification of A) 2 μm B) 100 μm;

FIG. 11 is an SEM image of zinc immersion coated AZ31 magnesium alloy at a magnification of A) 2 μmB) 100 μm;

FIG. 12 is an SEM image of copper coated AZ31 magnesium alloy at a magnification of A) 2 μmB) 10 μm;

FIG. 13 is an SEM image of iron coated. AZ31 magnesium alloy with an intermediate at a magnification of A) 2 μmB) 10 μm;

FIG. 14 is a graph of the XRD pattern of iron coated AZ31 with Fe 113 peak and MgO peaks;

FIG. 15 is a graph of the full XPS survey showing binding energies of different elements;

FIG. 16 shows graphs of XPS zoomed binding energy peaks for A) Carbon 1 s; B) Oxygen 1 s; C) Iron 2 p; and D) Magnesium 2 p;

FIG. 17 shows the calculation of coating roughness;

FIG. 18 shows an image of the roughness evaluation window evaluated using a VK-X100/X200;

FIG. 19 shows a 3D image of the surface coating of the substrate (magnesium body);

FIG. 20 shows a 3D image of the surface of the magnesium sample;

FIG. 21 shows a 3D top view of the magnesium substrate;

FIG. 22 shows an SEM image of the cross sectional view of iron coated AZ31 magnesium sample for thickness evaluation;

FIG. 23 is a graph of the Open Circuit potential comparison of the various coating approaches used;

FIG. 24 is a graph of the Potentiodynamic polarization curves;

FIG. 25 is an image of niobium coated magnesium; and

FIG. 26 is a graph of the biocompatibility of different organic coatings as compared to bare magnesium substrate.

### DETAILED DESCRIPTION

The present invention is concerned with implantable devices, and particularly magnesium-based implants, which are suitable for use in biodegradable (temporary) implant structures. Thus, the present application relates to biodegradable materials useful for manufacturing implantable medical devices, specifically biodegradable compositions comprising metal alloys that can provide high strength when first implanted and are gradually eroded (bio-corroded) and replaced with body tissue. The term “magnesium-based” means that the principle metallic element is magnesium, although other alloying elements (metals) may be present so long as magnesium levels are greater than about 80% and preferably greater than about 90% by weight.

The implantable device comprises generally a body formed at least in part by metallic magnesium or an alloy thereof. That is, one or more structural members of the device body may be formed all or in part by metallic magnesium or a magnesium-based alloy, such that parts of the implant are magnesium-based, while other parts may be formed of other suitable implant materials (e.g., plastics, stainless steel, composites, as well as biologic tissues/grafts, etc.). For example, in certain complex implants, only certain structural members of the implant may be magnesium-based (e.g., pins, screws, flaps, etc.). Likewise, the entire implant body can be formed of metallic magnesium or a magnesium-based alloy. This may be, although is not required to be, the case for implants such as screws, staples, stents, and the like. In one or more embodiments, the part(s) of the implant body formed by metallic magnesium or an alloy thereof can consist essentially or even consist entirely of the metallic magnesium or an alloy thereof.

As used herein, “implants” are devices introduced or inserted into the body of a patient or subject by invasive or non-invasive surgical methods, and include, without limitation, fastening elements for bones (e.g., pins, nails, screws, anchors, plates), surgical suture materials (e.g., staples, wire, metallic thread), clamps (e.g., intestinal), vascular or vessel clips, cardiovascular prostheses (e.g., for hard or soft tissue, such as stents and anchoring elements for electrodes for pacemakers and/or defibrillators), general stents, craniomaxillofacial reconstruction plates or fasteners, dental implants, vertebral body implants, porous bone graft scaffolds, valves, and the like. As such it will be appreciated the implants can be of various size and shape configurations.

Magnesium is a desirable implant material, particularly for temporary implants, because it is biocompatible and has a high strength-to-weight ratio, but degrades in the body with low toxicity side effects. Pure metallic magnesium is rarely used in physiological applications because of its reactivity and low corrosion resistance. Magnesium corrodes rapidly in a chloride atmosphere and in low pH containing acidic solution. Corrosion of magnesium in human body is due to high chloride concentration in blood and varying human body temperature. Magnesium corrosion resistance can be achieved by alloying and/or surface modification. Magnesium can be alloyed with metals like aluminum, calcium, steel, zinc, manganese, silicon, copper, rare earth metals (Li, Zr, Y) and zirconium to form strong alloys. It will be appreciated that as the implant biodegrades, the alloying elements will be released in the body; thus, the biocompatibility of selected alloying elements must be taken into consideration in selecting suitable magnesium-based material. An exemplary magnesium alloy for use is AZ31, which contains 3% aluminum, 1% zinc, 0.3% manganese, and balance magnesium.

Magnesium undergoes different types of corrosion in the presence of physiological media, including: I) galvanic corrosion; 2) stress corrosion; 3) pitting corrosion; and 4) fretting corrosion. The corrosion and degradation of magnesium-based materials in a physiological environment is termed “biocorrosion.” Galvanic corrosion is the one which takes place when two metals are electrically in contact through an electrolyte. In most of the cases magnesium acts a sacrificial anode. Probable ways to reduce galvanic corrosion is by minimizing the potential difference between magnesium and other metals and by maximizing circuit resistance. Stress corrosion is the occurrence of cracks in a corrosive environment. Stress corrosion can lead to sudden failure of metals. Pitting corrosion is the most the common form of corrosion found in magnesium and its alloys. This is a form of localized corrosion. Pitting takes place when magnesium is exposed to chloride or acidic medium. Pitting is accelerated in the human body due to changes in temperature. In pitting corrosion the pit acts as the anode and the entire surface of the metal acts as cathode; when there is a change of potential surface erosion takes place. Fretting corrosion occurs when two contacting pieces of metal are pressed by an external force, such that one gets displaced by less than 0.03 inch. Fretting is common in orthopedic applications.

Thus, although alloying reduces the corrosion rate of magnesium, magnesium alloys are still rapidly biodegradable under physiological conditions. Surface modification and coating is used according to the invention to increase the corrosion resistance of magnesium-based materials. Different coating techniques such as vapor deposition (chemical or physical) and electroplating can be used to obtain better corrosion resistance.

In more detail, the implant body formed at least in part by metallic magnesium or an alloy thereof has an external surface. The term “external surface” refers to the surface(s) of the implant body, which would come into contact with the physiological environment when implanted unless covered by a coating or film (i.e., the contacting surfaces of a bare implant). Thus, depending on the configuration and shape of the implant body, the external surface of the body may include both an outer surface and an inner surface, such as in the case of a hollow tubular body, where both the inner surface of the tube and the outer surface of the tube would be considered “external” surfaces with which physiological fluids, tissue, etc. may come into contact when the device is implanted into a patient or subject body.

At least a portion of the external surface is covered with a biocorrosion-inhibiting film. Thus, the biocorrosion-inhibiting film is a thin coating or layer of material adjacent at least a portion of the external surface to protect the external surface from biocorrosion when in contact with physiological conditions. As noted above, the implant by is formed at least in part by metallic magnesium or an alloy thereof. When the implant is composed of the biodegradable magnesium-based material only in part, the external surface of this part, in particular, should be appropriately coated with the biocorrosion-inhibiting film. That is, it will be appreciated that depending upon the material composing the other part(s) of the implant body (i.e., the non-magnesium-based parts or structural elements), it may not be necessary or desirable to cover the external surface of such other materials with the biocorrosion-inhibiting film.

Advantageously, the biocorrosion-inhibiting film comprises a metal. In one or more embodiments, the biocorrosion-inhibiting film consists essentially or even consists of metal. In one or more embodiments, the metal is biodegradable. The term “biodegradable,” as used herein, is synonymous with bioresorbable or bioabsorbable, and means that the material is one that can readily dissolve, corrode, or be absorbed under physiological conditions over time (e.g., in a human or non-human animal body, in vivo, or under in vitro physiological culture conditions of temperature and pH, etc.). Although readily degradable in vivo, suitable biodegradable metals are preferably selected such that they have a relatively slower biocorrosion rate in vivo than the magnesium-based material used to form the implant body. In one or more embodiments, the metal is non-biodegradable. It will be appreciated that the biocorrosion-inhibiting films should be thin, such that even non-biodegradable metals may eventually corrode and breakdown (but will not necessarily dissolve or be absorbed). Thus, residual metal fragments or particles of such non-biodegradable metals can still eventually be removed by the body's normal disposal systems (e.g., through phagocytosis, the lymphatic system, etc.). It will also be appreciated that the thickness of the biocorrosion-inhibiting film can vary widely and will be selected based upon the biocorrosion rate of the selected metal film, and the length of time the implant is desired to remain intact (i.e., maintain structural and mechanical integrity) before degrading within the patient or subject body. Accordingly, although the biocorrosion-inhibiting film reduces the rate of biocorrosion of the underlying magnesium-based implant material, it is not intended to completely prevent biocorrosion, but rather control the rate of corrosion to extend the life of the temporary implant to the appropriate time frame for the particular implant.

Preferably, the selected metal is biocompatible, which means that it is suitable for intimate contact with, and has minimal deleterious effects (e.g., toxicity) on living tissue. In one or more embodiments, the metal is selected from the group consisting of iron, niobium, titanium, tantalum, zinc, copper, cobalt, chromium, nitinol, combinations thereof, and alloys thereof. The biocorrosion-inhibiting film can be applied and/or affixed to the external surface of the implant body using any suitable technique. In one or more embodiments, the biocorrosion-inhibiting film is electroplated onto the external surface of the implant body, and particularly the magnesium-based parts of the implant body. The biocorrosion-inhibiting film can cover substantially all of the external surface of the magnesium-based parts of the implant body. Alternatively, the biocorrosion-inhibiting film may cover only selected portions of the external surface. This can be accomplished, for example, by selectively depositing the biocorrosion-inhibiting film onto discrete areas of the external surface. Similarly, the biocorrosion-inhibiting film can be a porous film, such that portions of the external surface remain uncovered via the pores in the film. That is, in one or more embodiments, the biocorrosion-inhibiting film comprises one or more openings or pores extending through the thickness of the film, such that the external surface is exposed or uncovered. Thus, the “exposed areas” of the external surface correspond to surface area of the body that is not covered by the corrosion-inhibiting film (or another layers as explained below), and is thus exposed and susceptible to the physiological conditions.

In one or more embodiments, the implantable device further comprises an optional intermediate film between the external surface of the implant body and the biocorrosion-inhibiting film. That is, the intermediate film is applied underneath the biocorrosion-inhibiting film. The intermediate film can be deposited using any suitable technique. Preferably, the intermediate film is biocompatible. In one or more embodiments, the intermediate film comprises a metal, and preferably a biocompatible metal. In one or more embodiments, the intermediate film consists essentially or even consists of metal. In one or more embodiments, the metal is biodegradable. In one or more embodiments, the metal is non-biodegradable. The metals can be selected from the group consisting of copper, iron, niobium, titanium, tantalum, zinc, cobalt, chromium, nitinol, combinations thereof, and alloys thereof. In one or more embodiments, the intermediate film can cover substantially all of the external surface of the magnesium-based parts of the implant body. Alternatively, the intermediate film may cover only selected portions of the external surface. In one or more embodiments, the intermediate film is non-porous. Thus, in embodiments where a porous corrosion-inhibiting film is used, it will be appreciated that portions of the intermediate film may be exposed via the pores in the corrosion-inhibiting film (instead of the external surface, which is correspondingly covered by the intermediate film). Thus, the “exposed areas” of the intermediate film correspond to surface area of the intermediate film that is not covered by the corrosion-inhibiting film.

In one or more embodiments, the implantable device can further comprise an optional adhesion promoting film between the external surface and intermediate film. That is, the adhesion promoting film is underneath the intermediate film and adjacent at least portions of the external surface of the implant body. The adhesion promoting film can be deposited using any suitable technique. Preferably, the adhesion promoting film is biocompatible. In one or more embodiments, the adhesion promoting film comprises a metal, and preferably a biocompatible metal. In one or more embodiments, the adhesion promoting film consists essentially or even consists of metal. In one or more embodiments, the metal is biodegradable. In one or more embodiments, the metal is non-biodegradable. The metals can be selected from the group consisting of zinc, copper, iron, niobium, titanium, tantalum, zinc, cobalt, chromium, nitinol, combinations thereof, and alloys thereof. In one or more embodiments, the adhesion promoting film can cover substantially all of the external surface of the magnesium-based parts of the implant body. Alternatively, the adhesion promoting film may cover only selected portions of the external surface. In one or more embodiments, the adhesion promoting film is non-porous. In embodiments where a porous corrosion-inhibiting film is used, it will be appreciated that the intermediate film may be exposed via the pores in the corrosion-inhibiting film, as described above. As the intermediate film biocorrodes, it will reveal exposed areas of the adhesion promoting film, which correspond to the surface area of the adhesion promoting film that is no longer covered by the intermediate film.

It will also be appreciated that the thickness of the intermediate film and/or adhesion promoting film can vary widely and will be selected based upon the biocorrosion rate of the selected metal film, and the length of time the implant is desired to remain intact (i.e., maintain structural and mechanical integrity) before degrading within the patient or subject body.

In one or more embodiments, the implantable device can further comprise a functional coating on the biocorrosion-inhibiting film. That is, in the device a functional coating or layer can be adjacent the biocorrosion-inhibiting film. It will be appreciated that where the biocorrosion-inhibiting film is porous, the functional coating may also be present in and over the pores in the biocorrosion-inhibiting film (i.e., it may coat the side walls of the pores and bottom wall of the pores, which may correspond to the external surface or intermediate layer, as applicable). In one or more embodiments, the functional coating can include therapeutic coatings, drug-eluting layers, synthetic or organic materials, combinations thereof, and other types of coating materials depending upon the implant. In one or more embodiments, the functional coating comprises one or more organic materials, such as organic polymers (e.g., polyesters, etc.). In one or more embodiments, the functional coating comprises one or more of active agents, such as therapeutic agents (e.g., biologics and/or small molecule drugs), antioxidants, anesthetic agents, anti-proliferative agents, osteoinductive agents, osteoconductive agents, and the like. Therapeutic agents for use in the invention include small molecule drugs as well as biologics. Exemplary small molecule drugs include antibiotics (e.g., vancomycin, tobramycin, gentamicin, nanoparticulate silver), anti-inflammatories (e.g., COX-1, COX-2, steroidals), and anti-coagulants (e.g., conjugated heparins, warfarin). Exemplary therapeutic biologics include proteins isolated from animal tissues such as collagen, albumin, fibrin, fibrinogen, vitronectin, and immunoglobulins, as well as synthetic, therapeutic monoclonal antibodies. Osteoinductive and osteoconductive agents (collectively osteopromotive) include allogenic or autogenic bone fragments, calcium phosphate, hydroxyapatite, bone morphogenetic proteins (BMP, isoforms 2, 4 and 7), cytokines and growth factors. The active agent can be immobilized on the biocorrosion-inhibiting film using any suitable technique. In one or more embodiments, the active agent can be dispersed in a suitable matrix or carrier that is biocompatible, and then applied to the biocorrosion-inhibiting film.

It will be appreciated that the implantable device can be introduced into a patient or subject body using the appropriate surgical technique. The magnesium-based implantable device is resistant to biocorrosion, but eventually breaks down and is biodegraded in the body as the hard and/or soft tissue repairs itself and ultimately replaces or resorbs the implant. Thus, the inventive coating system permits the use of magnesium-based materials for implants, while overcoming the drawbacks of the reactivity and low corrosion resistance of conventional magnesium implant materials.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

### 1. Materials Used

The substrate material was magnesium AZ31 alloy with a size of 35×35 mm size (Goodfellow Cambridge Limited). AZ31 contains 3% aluminum, 1% zinc, 0.3% manganese, and the rest magnesium. A magnesium alloy with low Al concentration like AZ31 is preferred for most biocompatibility tests. The corrosion rate of AZ31 magnesium alloy is higher than pure magnesium. AZ91 (9% Al, 1% Zn) offers better corrosion resistance than AZ31 but the higher aluminum content is neurotoxic. Nickel can also be electroplated onto magnesium for increasing its corrosion resistance. The counter electrode was an iron rod of length 100 mm and diameter 16 mm. Copper plates were from Goodfellow Cambridge Limited. All the other chemicals were from Sigma Aldrich.

### Selection of Ionic Liquid

An ionic is liquid is a salt in which the ions are poorly coordinated. They remain in solvent phase at a temperature less than 1000° C. Any salt which melts without decomposing or vaporizing gives an ionic liquid. Ordinary liquids like water and gasoline are made up of neutral molecules whereas ionic liquids are made up of ions or short lived pairs. When an ionic liquid is cooled it forms an ionic solid. These ions help in conduction of electricity. Ionic liquids are commonly used as electrolytes and powerful solvents. Ionic liquids were introduced to replace acidic baths commonly used in electroplating processes to lower the pH. This also had the advantage of creating electrolytic baths that were environment friendly.

A mass loss test was conducted among a set of 6 ionic liquids to determine which least corroded the magnesium surface owing to the fact that most electrolytic baths are acidic. Mass loss analysis is where the percentage loss in mass is found based on the initial and final mass of the substrate after an immersion time. Mass loss analysis does not give the degradation rate but gives the percentage mass loss before and after immersion. The percentage mass lost is given by the formula ((Initial mass-Final mass)/Initial mass)*100.

The six ionic liquids considered were: 1) 1-Butyl-2,3 dimethylimidazolium tetrafluoborate; 2) 1-Butyl Virdinium Chloride; 3) Trimethyl phenyl ammonium bromide; 4) Cholene Chloride; 5) Cholene Hydroxide+ethylene Glycol; and 6) 1-ethyl-3 methylimidazolium chloride. The samples taken for this analysis were a 0.25 mm AZ31 magnesium alloy. The test was carried out in a 1M solution of the corresponding ionic liquid for a time interval of 2 hrs, 4 hrs, 6 hrs and 8 hrs. At the end of each time interval the samples were taken out of the ionic liquid, rinsed in DI water and finally air dried. After selecting the ionic liquid an electrolyte was prepared with ferrous ammonium sulfate salt. Ferrous ammonium sulfate was selected because the sulfate ions did not affect the magnesium surface unlike chloride ions.

### Iron Plating

Iron was electroplated directly on the surface of AZ31 magnesium alloy. The ionic liquid was selected based on the ionic liquid test conducted. 1-Butyl-2,3 dimethylimidazolium tetrafluoborate was the ionic liquid used. The entire electrolytic bath consisted of 1M 1-Butyl-2,3 dimethylimidazolium tetrafluoborate and 350 g/l of ferrous ammonium sulfate. The anode used was a pure iron rod and the cathode used was an AZ31 magnesium alloy sample. The electrodes were connected to a D.C power and source and immersed in the electrolyte to complete a circuit. The entire experiment was carried out in a glass beaker. The experiment was carried out at a current density of 4 Adm⁻² for 20 minutes and the pH was maintained at 2-4.

TABLE
Contents and parameters for electroplating
iron directly on AZ31 magnesium

| Anode | Pure iron rod |
|---|---|
| Cathode | AZ31 Magnesium Alloy |
| Ionic Liquid | 1-Butyl-2,3dimethyIimidazolium |
| | tetrafluoborate |
| Electrolyte | Ferrous Ammonium Sulfate |
| Current Density | 4 Adm⁻² |
| Time | Variable based on coating thickness desired |
| pH | 2-4 |

### Iron Plating With an Intermediate Layer

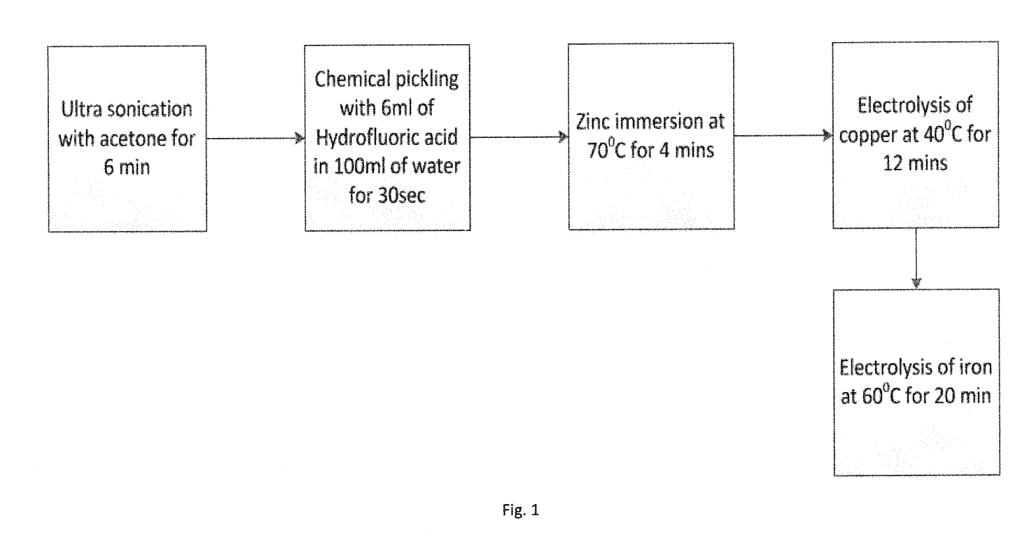

Iron was electroplated on the surface of the magnesium alloy by depositing a set of intermediate layers on the magnesium surface to prevent magnesium interaction with the electrolyte. The electroplating process was done in 5 steps: 1) Ultrasonication; 2) Chemical pickling; 3) Zinc immersion; 4) Copper plating; and 5) Iron plating, illustrated in FIG. 1.

### Ultrasonic Cleaning

Ultra sonication process is carried out in an ultra sonication instrument which produces sound waves with an appropriate solvent to clean the surface. The solvent usually depends on the type of the application. Cavitation bubbles induced by high frequency sound waves are used to agitate the cleaning solution. Aim of this test if to remove dust, dirt, pigments, rust, grease and bacteria. In this process acetone was used for cleaning. Acetone was selected because it is a good disinfectant, highly miscible with water and the flash point is much higher compared to other alcoholic solvents commonly used. The process was carried out for 6 min.

### Chemical Pickling

Chemical pickling is a method used to dissolve oxide layer on the surface of substrate. A solution called pickling liquor is used for this process which commonly contains a strong acid. Some commonly used acids are hydrochloric, sulfuric and hydrofluoric acid. Hydrochloric acid was not selected, because magnesium undergoes heavy corrosion in chloride based mediums. Hydrofluoric acid was selected because magnesium corrodes comparatively less in fluoride based medium. The magnesium fluoride layer formed on the surface of AZ31 offered better corrosion resistance and helpful for growth of osteoblast cells.

In this process a very dilute solution for etching was made. The solution consisted of 6 ml hydrofluoric acid in 100 ml of water. The process was carried out for duration for 30 sec and then rinsed in DI water.

### Zinc Immersion

An adherent coating of zinc is obtained on the surface of magnesium only if it is activated by an acidic bath or an alkaline bath. An aqueous zinc immersion bath usually consists of a pyrophosphate, a zinc salt, a fluoride salt and some quantities of carbonate if needed to maintain the pH. The zinc immersion layer ensures better adhesion of electroplated copper on the surface of magnesium. Presence of this zinc layer ensures that the magnesium substrate does not interact with the copper electrolyte. The process starts by immersion of zinc sulfate in DI water and then potassium pyrophosphate is added so that potassium complexes with zinc to form potassium zincate. Formation of the complex compound was indicated by the chemicals dissolving in DI water. Then potassium fluoride was added and then finally sodium carbonate was added.

The bath was operated at a·pH of 10.0+2 and at a temperature of 70° C. Time of immersion varied between 3.5 to 5 min. Immersion times depend on the Al content of the alloy.

TABLE
Contents and conditions of zinc immersion bath

| Zinc sulfate heptahydrate | 30 | g/l |
|---|---|---|
| Potassium Pyrophosphate | 180 | g/l |
| Potassium Fluoride | 5 | g/l |
| Sodium Carbonate | 5 | g/l |
| pH | 10.0 ± 2 | |
| Temperature | 70° | C. |

### Copper Electroplating

A layer of copper was applied on to the surface of zinc coated magnesium by electrolysis for 12 min. The bath was prepared by dissolving potassium pyrophosphate in DI water and copper pyrophosphate was dissolved in it forming a complex compound potassium cuprate. Finally ammonium citrate was added. Copper electroplating can be done from a variety of salts like copper sulfate, copper cyanide, copper fluroborate and copper pyrophosphate. Copper pyrophosphate was selected because the bath can be operated at an alkaline pH whereas copper sulfate and fluroborate cannot be operated at high pH.

TABLE
Contents and conditions of copper electroplating bath

| | | |
|---|---|---|
| Copper Pyrophosphate | 28 | g/l |
| Potassium Pyrophosphate | 100-150 | g/l |
| Ammonium Citrate tribasic | 60-70 | g/l |
| Time of plating | 12 | min |
| Temperature | 40° | C. |
| Current Density | 1.2 | A/dm² |

| | |
|---|---|
| pH | 8.2-8.8 |
| Anode | Copper rod |
| Cathode | Zinc immersion coated magnesium |

Iron Electroplating

A commonly used double salt, ferrous ammonium sulfate is used for electrolysis purpose. The bath can be operated from 25° C. to 60° C., rate of deposition increases with increasing temperature but there is no change in rate beyond 60° C. Addition of ammonia to ferrous sulfate can reduce the rate of oxidation and prevent formation of iron oxide in the bath. The entire set up consists of an anode which is an iron rod and cathode is copper coated magnesium. The current density was maintained at 4 dm⁻².

TABLE
Contents and conditions of iron electroplating process

| Ferrous Ammonium Sulfate | 350 | g/l |
|---|---|---|
| 1-Butyl-2,3dimethylIimidazolium | 12.5 | g/l |
| tetrafluoborate | | |
| Temperature | 50° | C. |
| pH | 2.5 | |
| Time | 25 | min |
| Current density | 4-10 | A/dm² |

| | |
|---|---|
| Anode | Iron rod |
| Cathode | Copper electroplated magnesium |

### Summary

The entire process parameters and conditions from beginning to end is summarized in the following table

TABLE
Contents and conditions of the entire iron plating process

| Process | Solution composition | | | Conditions | |
|---|---|---|---|---|---|
| Ultra sonic | Acetone | | | 6 | min RT |
| cleaning | | | | | |
| Pickling- | HF(40%) | 6 | ml/l | 30 | s RT |
| Activation | | | | | |
| Zinc immersion | ZnsO₄•7H₂0 | 30 | g/l | 4 | min |
| | K₄P₂O₇ | 180 | g/l | 70° | C. |
| | KF | 5 | g/l | pH 10.0 ± 0.2 | |
| | Na₂CO₃ | 5 | g/l | | |
| Copper plating | | | | 12 | min |
| | Cu₂P₂O₇ | 28 | g/l | 40° | C. |
| | K₄P₂O₇ | 150 | g/l | pH = 8.2-8.8 | |
| | (NH₄)₃C₆H₅O₇ | 70 | g/l | J = 1.2 A/dm² | |
| Iron plating | | | | 25 | min |
| | (NH₄)₂Fe(SO₄)₂•6H₂O | 350 | g/l | pH 2.5 | |
| | C₉H₁₇BF₄N₂ | 12.5 | g/l | 50° | C. |

J = 4-10 A/dm²

### Characterization Techniques

The surface morphology was observed using a Zeiss scanning electron microscope. Characterization was done using Leica optical microscopy which showed the surface of samples at different magnifications. XRD also reports the elemental composition but at the same time gives an idea about bond strength.

### Optical Microscopy

The Leica Optical Microscopy connected with a digital camera and a computer was employed for viewing the surface of the coated samples. In order to process the images Leica vision was installed on the computer. Depending on the demands of the experiment different magnifications can be obtained. An inverted microscope (Optical microscope apparatus is normally used to view metal surface due to the absence of an inverted microscope a flash light was used to mimic an inverted microscope. Images were taken at 10×, 20×, 30× magnification.

### SEM-EDX

The scanning electron microscope has a large vacuum tube in which electrons are produced by a heated filament and they are driven by high voltage finally the electrons are made to strike the sample which produces an image. Mostly conductive materials can only be imaged if it is not conductive it can be made conductive by coating with a conductive material. This procedure enables higher magnification compared with optical microscope and the image obtained from a scanning electron microscope has a greater field depth. They have little features which can be imaged clearly. In addition to scanning electron microscopy, they generate x-rays which gives the elemental composition of the surface. EDX can be used in determining the elemental composition of metals heavier than boron not including hydrogen. The size of the sample which can be used for elemental detection is limited to a few cubic inches.

### X-Ray Diffraction

X-ray diffraction is a method in which the atomic and molecular structure of a crystal can be determined. Different kinds of crystal can cause x-rays to deflect in a particular pattern. The kind of element can found out by comparing the peaks with the already present reference metals. XRD used is divided into single crystal and poly crystalline. Since iron coated sample with an intermediate layer has more than one kind of element a polycrystalline XRD was preferred. The XRD set consists of a goniometer emitting x-rays, which is made to strike the sample at an angle. The reflection gives two dimensional images which is converted into 3 dimensional electron density, using the mathematical method of fourier transforms, combined with chemical data known for the sample, the patterns can be obtained. A Polycrystalline Bruker D8 Discover X-Ray Diffraction Machine was used in these tests.

### X-Ray Photoelectron Spectroscopy

X-ray photoelectron spectroscopy (XPS) is a quantitative spectroscopic technique which calculates the elemental composition, empirical formula, chemical state and electronic state. In this case XPS was performed just for determining the elemental composition. It is a technique where the surface of the substrate can be analyzed. XPS detects all elements higher than lithium but it cannot detect hydrogen or helium. Detection limit for XPS is mostly in parts per thousand, detection in parts per million is obtained but it needs special conditions. XPS values are obtained by making the X-rays strike the surface of the substrate and measuring the kinetic energy and the number of electrons that escape from the top 1 to 10 nm of the material. This process is done at ultra high vacuum. XPS analysis used was a large area mode (7*20 mm) and the anode used was aluminum at 250 W and the pass energy used was 20. Three scans were taken at 1 eV/step.

### Roughness Evaluation

Roughness of the iron coated sample was evaluated using a VK-X100/X200. It is used to perform non-contact profile and roughness measurements on any metal. It is a combination of an SEM, microscope and roughness gauge. Poor resolution, shallow depth and traceability are some disadvantages with microscope. Monochrome, time consuming, limited sample size are SEM's disadvantages. This instrument is combination of all advantages all these instruments and eliminates all of their disadvantages. This instrument is similar to a non contact angle type profilometer. Line scans are performed all over the sample to find the corresponding surface roughness. The images were taken on the iron coated sample. All the images were taken at a magnification of 10×.

### Thickness Measurement

The thickness of the iron coated samples was evaluated using SEM. The sample used was a 0.25 mm (250 μm) thick AZ31 magnesium sample. The samples were dipped in liquid nitrogen; it makes the metal brittle to gives a clean break. A clean break is necessary to view the layers. The entire idea behind dipping the substrate in liquid nitrogen is to break the sample rather than shearing it.

### Corrosion Evaluation

Corroding ability of iron coated AZ31 magnesium alloy was evaluated by evaluated by and less porous coatings yield better corrosion resistance. Electrochemical methods give an idea of corrosion capability over a year. All the electrochemical tests were conducted in a Gamry Reference 600 ™ potentiostat/Galvenostat in phosphate buffered saline solution (PBS). PBS is a buffer solution which is commonly used in biological research. It is an aqueous solution containing salts like sodium chloride, sodium phosphate, potassium chloride and potassium phosphate. The ion concentrations of this solution match that of the human body. The electrochemical tests were conducted in a 3 electrode corrosion cell. The entire set up consisted of an anode, a cathode and a reference electrode which is a saturated calomel electrode. The voltage was swept between the reference electrode and the working electrode. The potentiodynamic curves and open circuit curves were obtained at a constant voltage scan rate of 10 mV/s. The specimen area exposed was 0.9503 cm² with scan voltage ranging from 2 to −2 V. The open circuit voltage actually tells how much the given sample would corrode without passing any current through it. The experimental parameters for tafel and open circuit potential is given below

TABLE
Experimental parameters for tafel plot and open circuit potential

| Initial Voltage | 2.5 V vs. Eoc | |
|---|---|---|
| Final Voltage | −2.5 V vs. Eoc | |
| Scan Rate | 10 | mV/s |
| Sample Period | 0.245 | s |
| Specimen Area | 0.9503 | cm² |

### 2. Results and Discussion

### Ionic Liquid Test

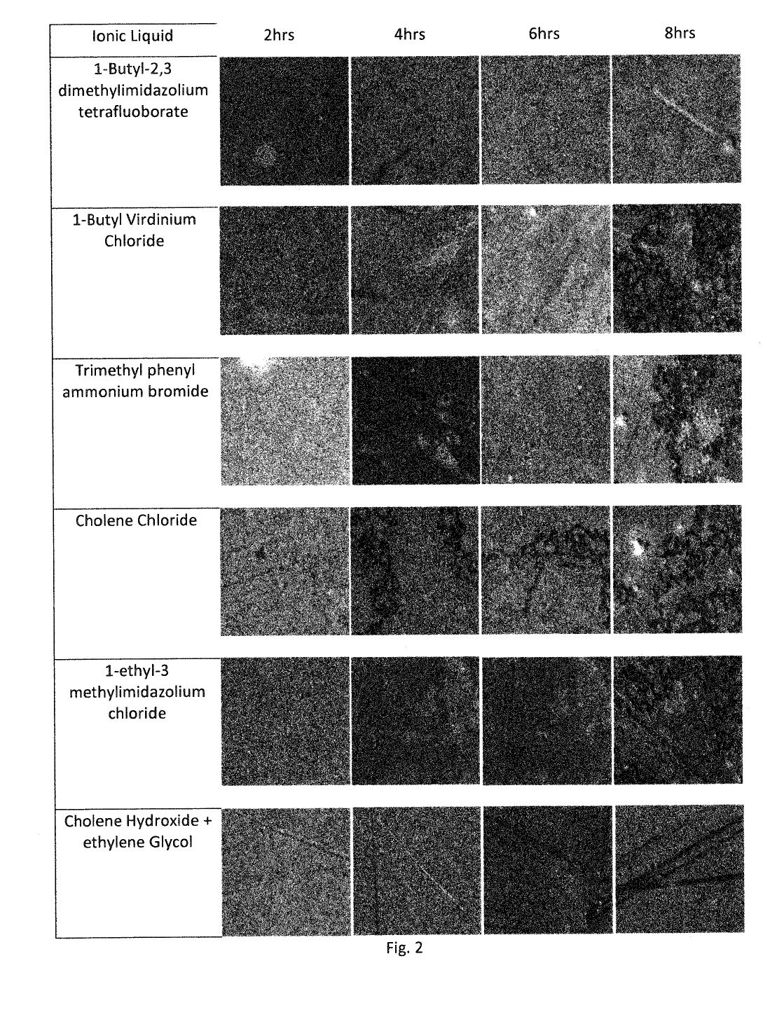

Based on the mass loss analysis tests conducted 1-Butyl-2,3 dimethylimidazolium tetrafluoborate and cholene hydroxide+ethylene glycol was found to be best with the lowest mass loss percentage. FIG. 2 shows the corrosion on the surface of the magnesium substrate for different ionic liquids. The samples dipped in 1-Butyl-2,3 dimethylimidazolium tetrafluoborate and cholene hydroxide was found to have the least corrosion. 1-Butyl-2,3 dimethylimidazolium tetrafluoborate was chosen among the two because cholene hydroxide+ethylene glycol mixture is highly basic and has a pH of 13 which drastically increases the pH of the electrolytic bath to 12 where iron precipitates as ferric hydroxide and lowers the throwing power of the bath.

TABLE
Mass loss analysis data of the ionic liquid test conducted

| | 0 hrs | 2 hrs | 4 hrs | 6 hrs | 8 hrs | % mass |
|---|---|---|---|---|---|---|
| Ionic Liquid | Weight (g) | Weight (g) | Weight (g) | Weight (g) | Weight (g) | loss |
| 1-Butyl-2,3 | 0.1015 | 0.1015 | 0.1013 | 0.1017 | 0.1015 | 0 |
| dimethylimidazolium | | | | | | |
| tetrafluoborate | | | | | | |
| 1-Butyl Virdinium | 0.1165 | 0.1158 | 0.1133 | 0.1128 | 0.1125 | 3.5 |
| Chloride | | | | | | |
| Trimethyl phenyl | 0.113 | 0.1123 | 0.1111 | 0.1103 | 0.1106 | 0.63 |
| ammonium bromide | | | | | | |
| Cholene Chloride | 0.1168 | 0.1148 | 0.1143 | 0.1131 | 0.1140 | 2.3 |
| 1-ethyl-3 | 0.1275 | 0.1260 | 0.1256 | 0.1252 | 0.1254 | 1.6 |
| methylimidazolium | | | | | | |
| chloride | | | | | | |
| Cholene Hydroxide + | 0.739 | 0.740 | 0.735 | 0.739 | 0.739 | 0 |
| ethylene Glycol | | | | | | |

### Iron Plating Without an Intermediate Layer

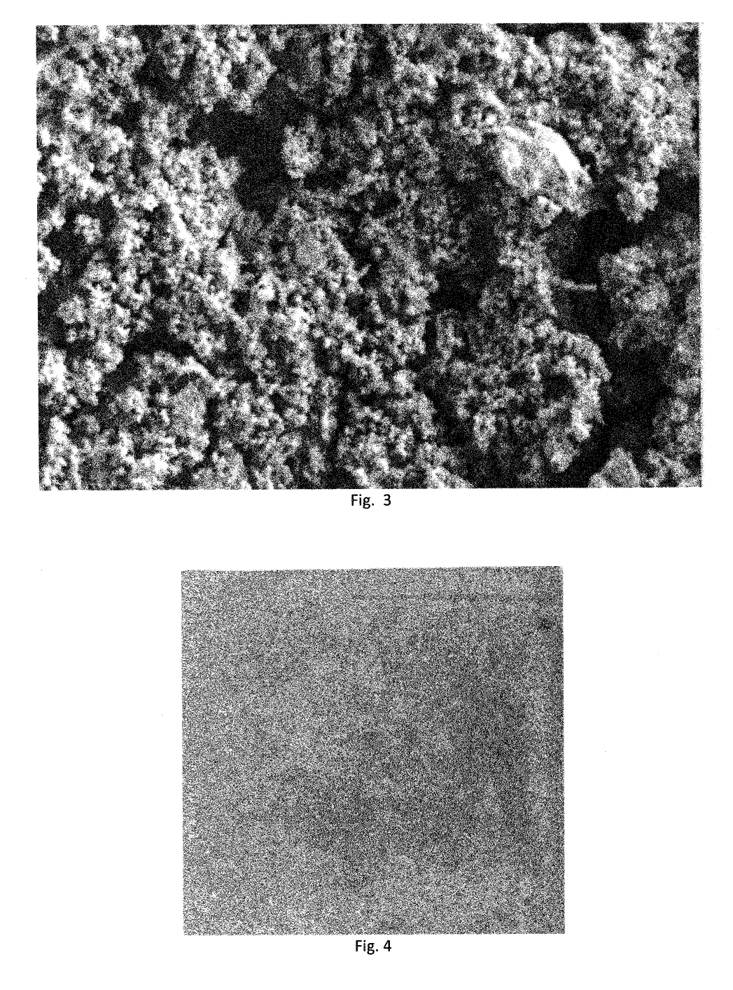

Iron was electroplated directly on the surface of magnesium without an intermediate layer. Since magnesium is highly reactive it corrodes at a pH less than 10. Magnesium undergoes pitting corrosion and the primary corrosion product is magnesium hydroxide which increases the pH of the electrolyte, and the pH increased from 2 to 4 during the plating time. Presence of hydroxide precipitates also lowers the throwing power of the bath which is its ability to plate without any pores. As a result of which, magnesium reacts with ferrous ions in the solution and oxidize them to ferric ions thereby making the bath sludgy. The deposits obtained on the surface of magnesium were ferric and immersion deposits which stick to the magnesium surface due its reactivity. Besides that, magnesium corroded during the plating period. The coating obtained was very porous and it formed a galvanic cell which increases the rate of corrosion. In this case, magnesium becomes the sacrificial anode and corrodes at a rate faster than usual. From FIG. 3, it is seen that the coating obtained is highly porous and highly oxidized which will be proved by EDX analysis.

### Iron Plating With an Intermediate Layer

Iron was electroplated on the magnesium surface as mentioned in the methods sections. The results and discussion of each and every stage is mentioned below.

### Chemical Pickling

The AZ31 magnesium alloy consists of two phases α (Mg) and β phase (Mg₁₇Al₁₂). The α phase consists of pure magnesium and β phase consists of magnesium and aluminum. After HF activation, the potential difference between the α and β phase is reduced, which contributes to the lower chemical reactivity of AZ31 magnesium alloy. After the initial HF activation for 30 sec the surface of the specimen gets etched at the β and α interface. The activation step not only dissolves the oxide layer but also stalls fierce replacement of oxide layer to an extent which is necessary to plate smooth layers.

### Zinc Immersion

The pyrophosphate added dissolves all oxides and hydroxides on the surface of the substrate to form water soluble complexes. Rate of deposition depends on the fluoride content, where a lower fluoride content causes a high rate of deposition which produces a loose coating. Amount of carbonate salt added depends on the pH. Water for preparing the bath needs to be free from iron deposits in order to get pore free deposits

A zinc immersion layer was coated on the surface of the HF pickled magnesium. EDX results showed no concentration of Al and very high concentration of Zn. The β phase was gradually dissolved and covered by a Zn layer during the time of immersion. The Zn layer will gradually extend to a phase and reduce the chemical reactivity of substrate. The potential difference between α and β phase is reduced at the end of the immersion process which makes the substrate more stable than the HF activated zinc. Zn layer also helps better adhesion of the intermediate copper layer. FIG. 4 shows the zinc immersion coated magnesium substrate.

### Copper Electroplating

After the zinc immersion layer, copper was electroplated on the surface of zinc. The zinc layer formed was resistant to any attacks in the electroplating bath but non-resistant to attacks by ferrous ammonium sulfate bath. Hence a layer of copper was deposited on to the zinc immersion coated magnesium.

Copper cyanide, copper pyrophosphate, and copper sulfate were evaluated for copper electroplating process. Copper cyanide was not selected owing to its toxicity. Copper sulfate was also not selected because it could be operated only at very low pH, while the zinc layer could not withstand acidic attacks at a pH of 2.

The primary aim of the copper layer was to prevent attack in acidic medium. Since electroplating of iron is done at a very low pH there is a need to make the magnesium surface non-reactive at a very low pH. During electroplating a high current density of 3-4 A/dm² is applied for a period of 1 min and then reduced to 1.2 A/dm². Care was taken so that the process was not carried out at a high current density during the period which results in burnt and powdery deposits.

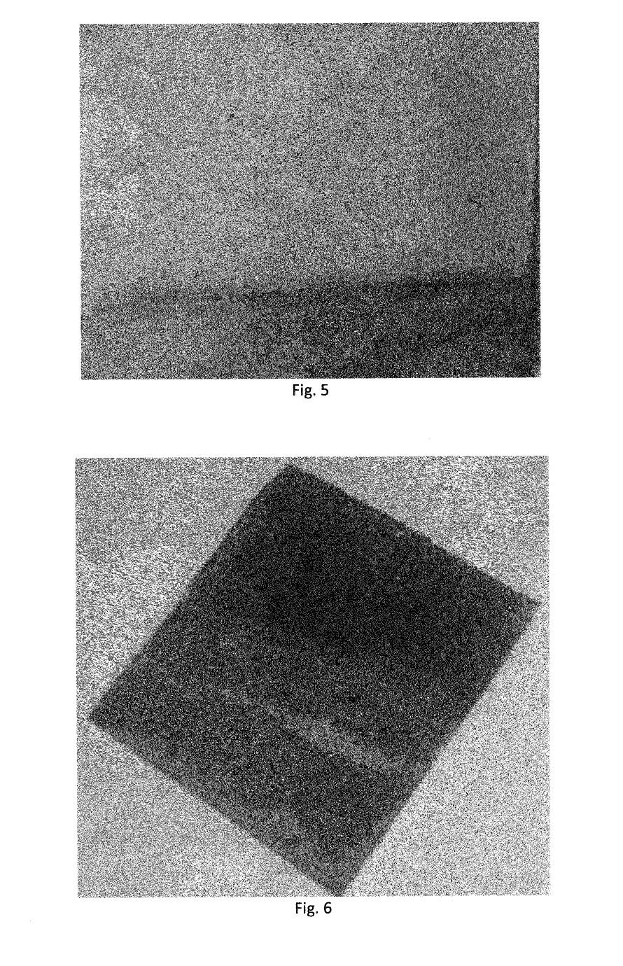

Gassing must be prevented during electroplating process which takes place when there is a high current flow when the part to be plated enters the bath. A voltage of 0.5 to 0.8V needs to be maintained initially to prevent gassing. Having a very high initial current density causes burnt and powdery deposits. The size of the cathode must be at least few times the size the anode. FIG. 5 shows the copper coated magnesium substrate

### Iron Electroplating

After a layer of copper was deposited on the surface of zinc immersed magnesium, iron was electroplated from a ferrous ammonium sulfate bath. Ferric chloride is considered to be the best bath for electroplating of iron, but since magnesium undergoes corrosion in a chloride medium it was rejected. FIG. 6 shows the camera image of iron coating with an intermediate copper layer. The coating obtained was grey and shiny.

### Optical Microscopy

The optical microscope images were taken for each and every layer. The images are listed in different levels of magnification for different layers.

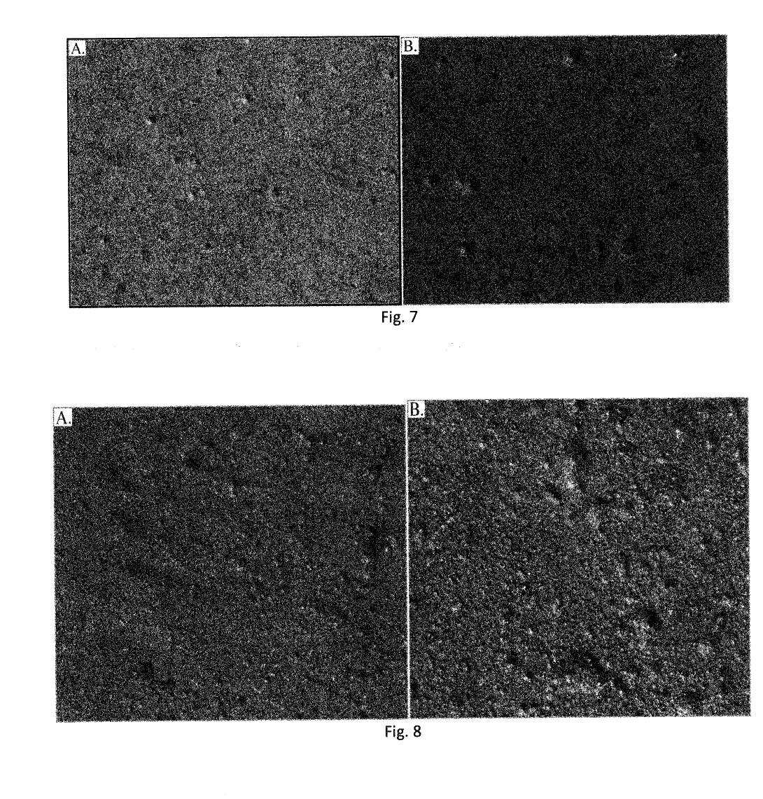

Zinc Immersion. The optical microscope images of zinc immersion coated magnesium at different magnification are shown. The magnesium surface was entirely covered and no pores were found on the surface of substrate. The optical microscope image of zinc immersion coated AZ31 magnesium alloy at a magnification of A)10× and B) 20× is shown in FIG. 7.

Copper Electroplating. The optical microscope image of copper plated AZ31 magnesium alloy at A)10× and B) 20× magnification is shown in FIG. 8. The coating was red and shiny. The coating had no pores on it.

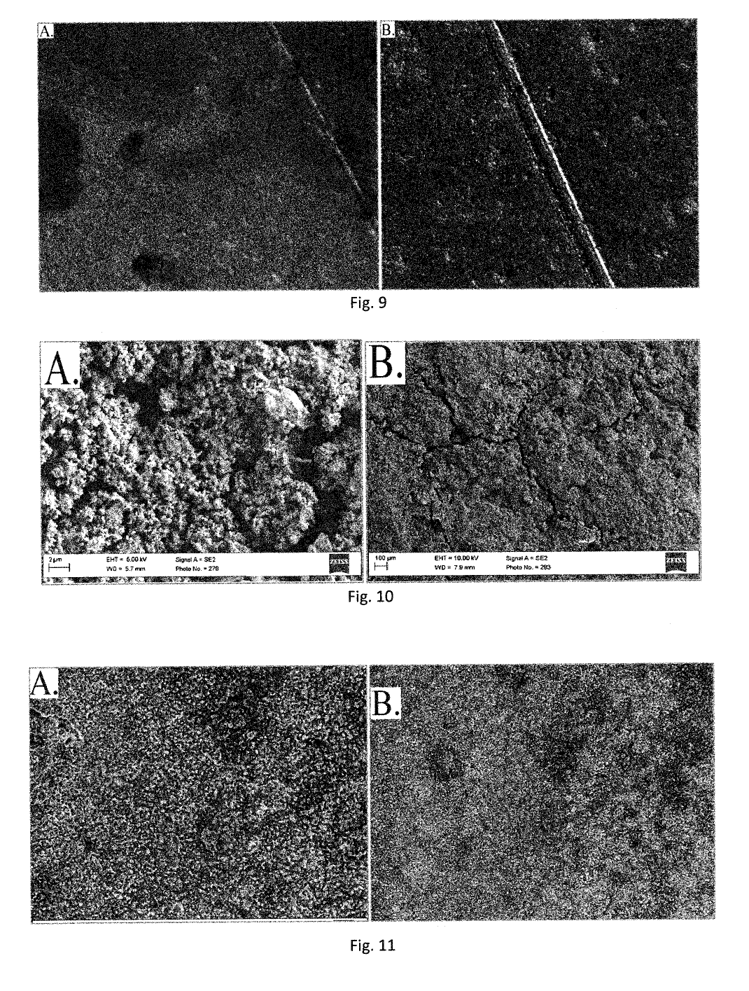

Iron Electroplating. Iron was electroplated on the copper coated magnesium. FIG. 9 shows the optical microscope image of iron electroplated AZ31 magnesium alloy with an intermediate copper layer at a magnification of A)10× and B) 20×. As shown in FIG. 9, the coating obtained was grey and shiny. The coating obtained was ferrous and the coating underwent less rusting compared to the iron coated magnesium without an intermediate sublayer.

### SEM-EDX

SEM-EDX analysis was conducted using the equipment as mentioned in the methodology. EDX analysis proves the presence of each and every element during different stages of the coating.

Iron Plating without an Intermediate Layer. As shown in FIG. 10, SEM image of iron coated magnesium without an intermediate layer shows that the coating is porous and further EDX analysis proved the less iron content and high oxygen content due to very high oxidation.

TABLE
EDX analysis of iron coated AZ31 magnesium
alloy without an intermediate layer

| Element | Weight % | Atomic % |
|---|---|---|
| Carbon | 16.12 | 28.79 |
| Oxygen | 34.89 | 46.52 |
| Magnesium | 4.09 | 3.61 |
| Phosphorus | 0.85 | 0.65 |
| Sulfur | 12.16 | 8.14 |
| Iron | 31.99 | 12.29 |
| Total | 100.00 | 100.00 |

Iron Coated Magnesium With an Intermediate Layer

The iron coated magnesium with an intermediate layer is done in three major steps. The SEM-EDX analysis of every step is given as follows.

Zinc Immersion. As shown in FIG. 11, SEM image of zinc coated magnesium shows a non-porous image and the magnesium surface was fully covered with the zinc immersion coating. EDX analysis shows the presence of zinc with traces of oxygen.

TABLE
EDX analysis of zinc coated AZ31 magnesium alloy substrate

| Element | Atomic % | Weight % |
|---|---|---|
| Oxygen | 4.34 | 14.37 |
| Magnesium | 4.96 | 10.80 |
| Phosphorus | 0.66 | 1.13 |
| Potassium | 1.46 | 1.97 |
| Zinc | 88.58 | 71.73 |

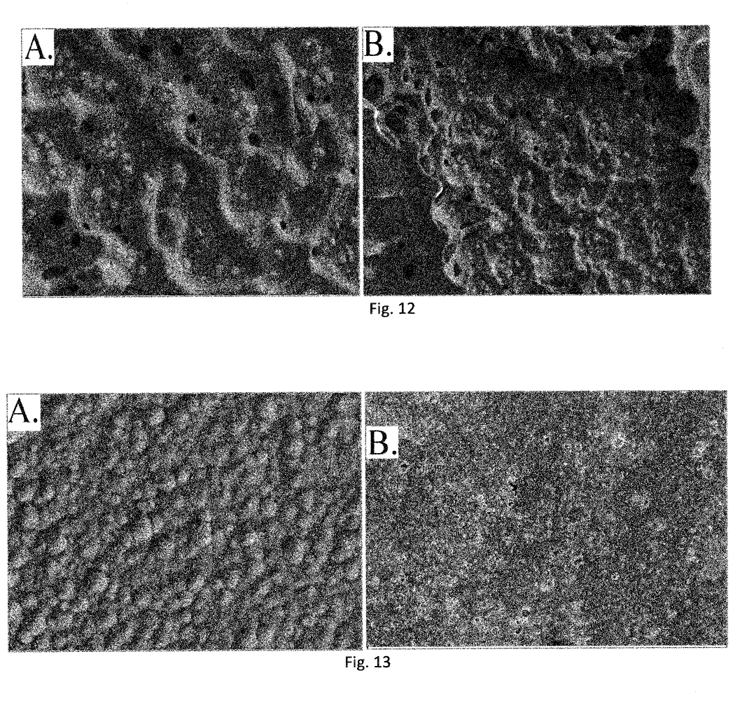

Copper Electroplating. As shown in FIG. 12, SEM image of copper coated magnesium shows a fully coated substrate with no pores and EDX analysis shows the presence of copper.

TABLE
EDX analysis of copper coated AZ31 magnesium alloy substrate

| Element | Atomic % | Weight % |
|---|---|---|
| Carbon | 4.15 | 15.98 |
| Oxygen | 5.88 | 16.97 |
| Phosphorus | 1.24 | 1.85 |
| Potassium | 1.50 | 1.77 |
| Copper | 87.23 | 63.43 |

Iron Electroplating. Iron was electroplated on the surface of copper coated AZ31 magnesium alloy. As shown in FIG. 13, the coating was less porous compared to the coating without an intermediate layer. EDX analysis confirmed the presence of 40% iron by atomic weight.

TABLE
EDX analysis of iron coated AZ31 magnesium
alloy with an intermediate layer

| Element | Weight % | Atomic % |
|---|---|---|
| Iron | 37.89 | 40.97 |
| Copper | 62.11 | 59.03 |

X-Ray Diffraction

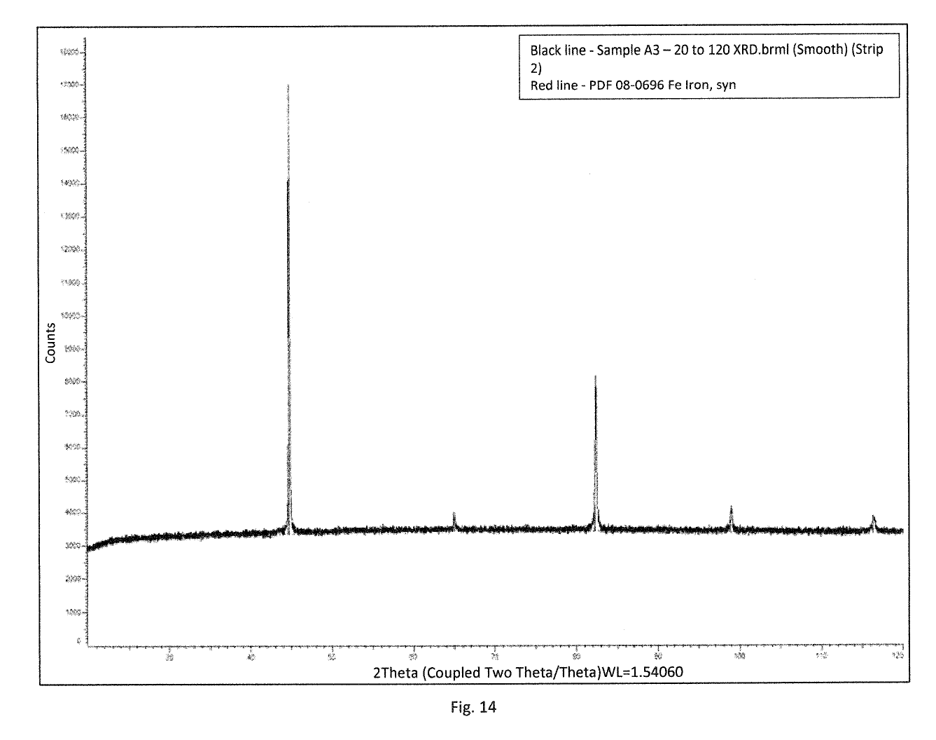

XRD data was collected using a bruker D8 discover system with a Cu X-ray tube. The samples were collected at 1600 W. The data was smoothed and the kα2 peak stripped prior to library matching. The scan was done from 20 to 120 to check for all possible elements present. The red line in FIG. 14, indicates the standard reference value of iron 113. The standard angle of diffraction for iron 113 is 42-43 degrees. The value of obtained is around 44 which confirms the presence of 113 iron. The iron present is in magnetite form. The XRD peak around 80 is the oxidized magnesium (MgO). The peak shows the presence of the AZ31 magnesium substrate. The peak at 65 is also a peak of magnesium.

### X-Ray Photoelectron Spectroscopy

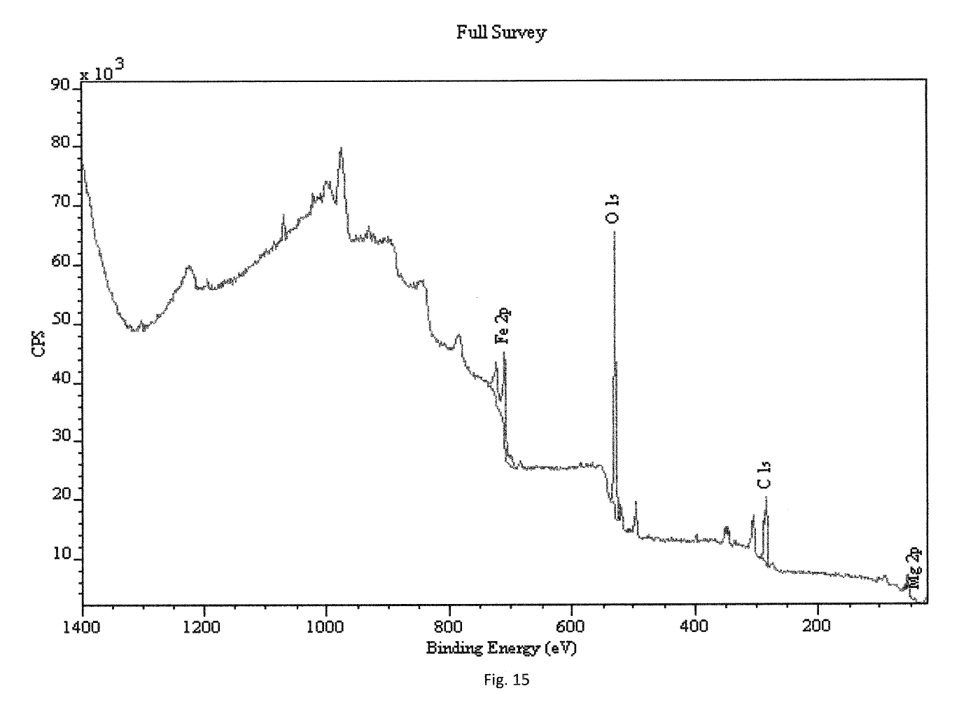

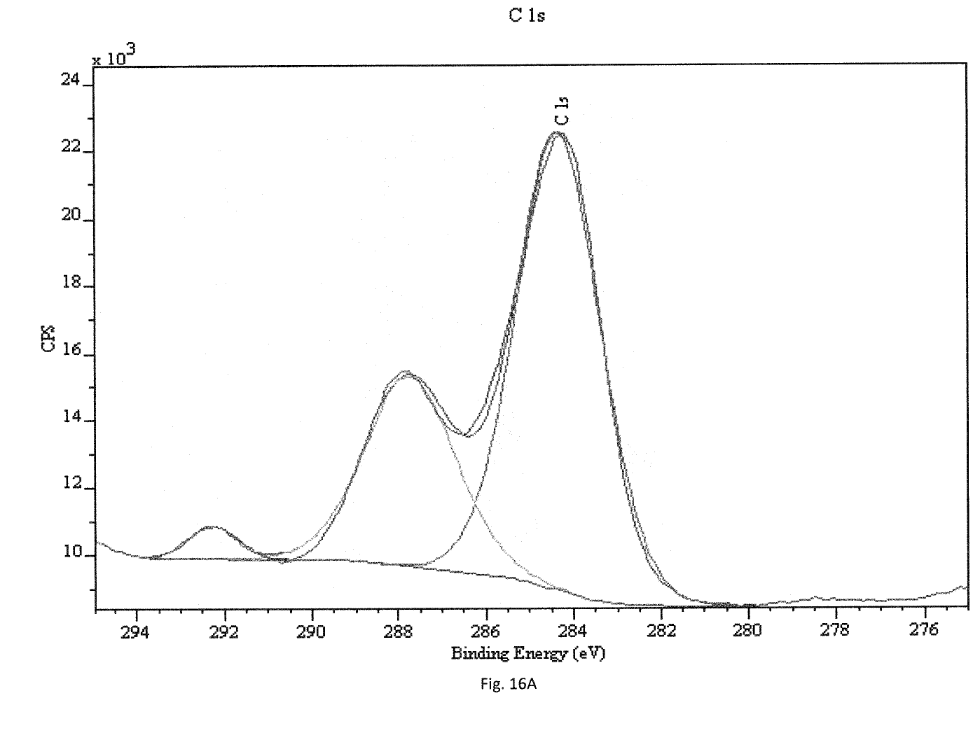

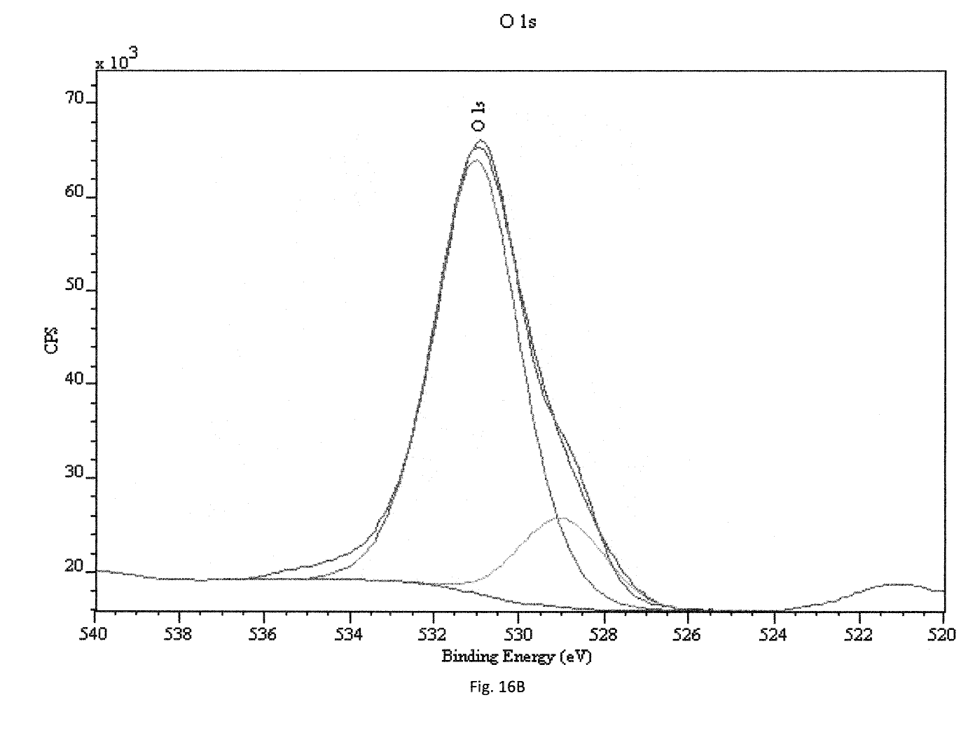

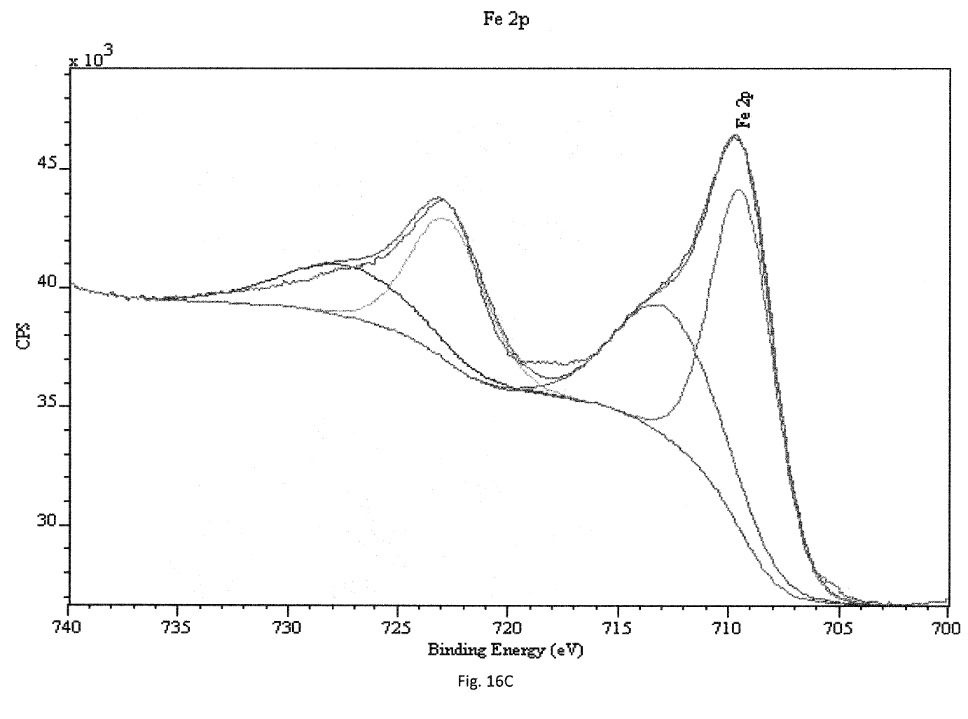

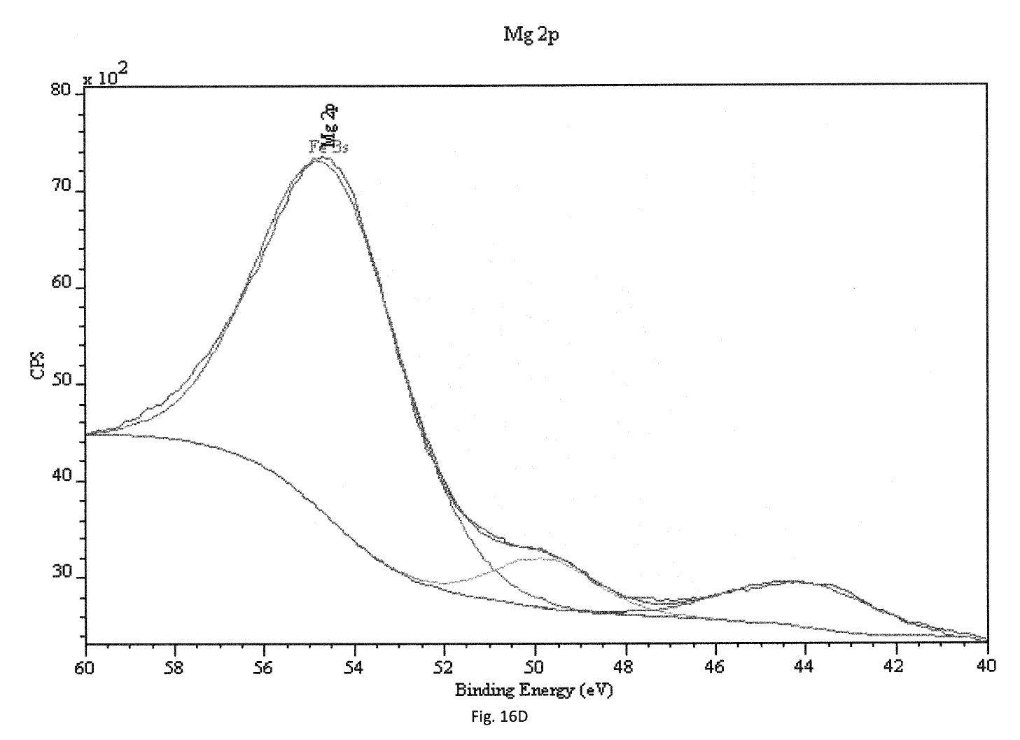

The basic idea of XPS is based on binding energy of each and every type of element. Binding energy is the least amount of energy required to keep the electron in the outer most orbit to be in its valance shell. Elemental composition is obtained based on when the electron escapes at the already found binding energy of each and every type of element. The Fe 2p region exhibits 2 moieties, with spin orbit splitting resulting in 4 peaks. As shown in FIGS. 15 and 16, the Mg region has peaks associated with the Fe 3 s peak at 53 eV.

TABLE
XPS analysis results for iron coated AZ31 magnesium
samples with an intermediate layer

| | Relative | | | | |
|---|---|---|---|---|---|
| | Sensitivity | | | Full Width at Half | |
| | Factors(RSF) | | Position | Maximum(FWHM) | Area |
| C 1s | 1 | Peak 1 | 284.333 | 2.25 | 33108 |
| | | Peak 2 | 287.728 | 2.45 | 15073 |
| | | Peak 3 | 292.272 | 1.22 | 1318.9 |
| O 1s | 2.93 | Peak 1 | 529.001 | 2.37 | 122151.2 |
| | | Peak 2 | 530.989 | 2.37 | 23839.9 |
| Fe 2p | 16.4 | Peak 1 | 709.337 | 3.34 | 51815.6 |
| | | Peak 2 | 712.754 | 4 | 32512.4 |
| | | Peak 3 | 722.703 | 5.52 | 25907.8 |
| | | Peak 4 | 727.291 | 7 | 16256.2 |

### Roughness Evaluation

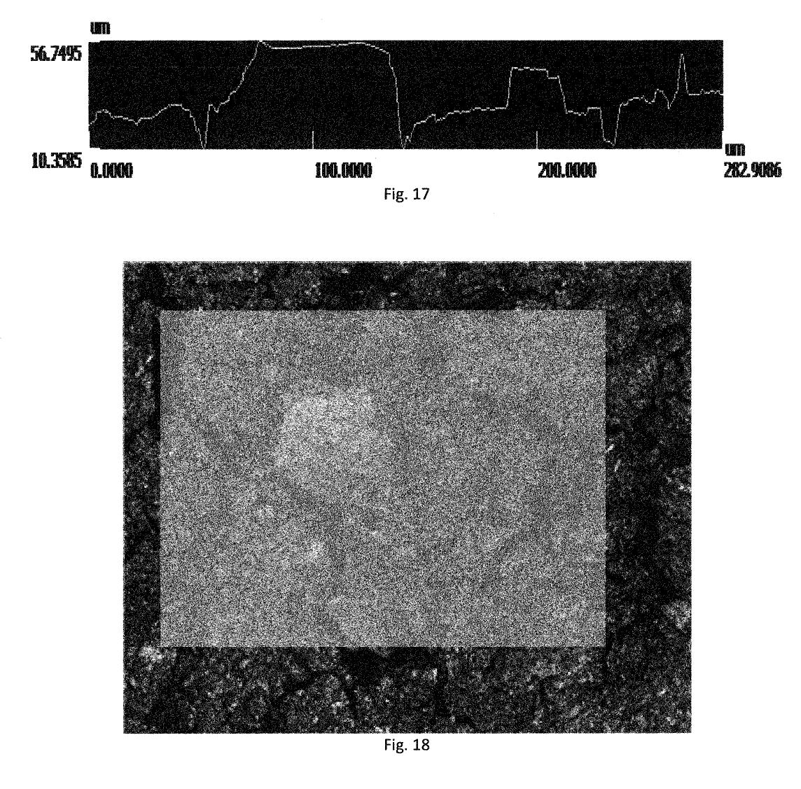

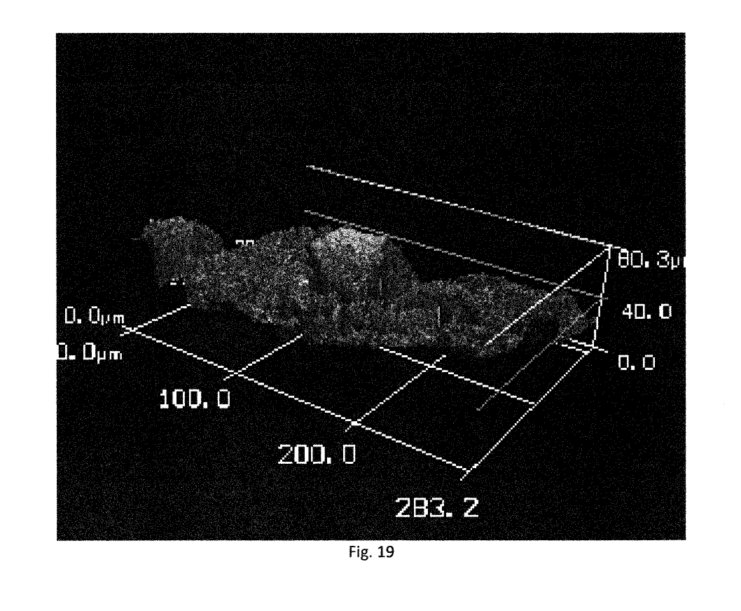

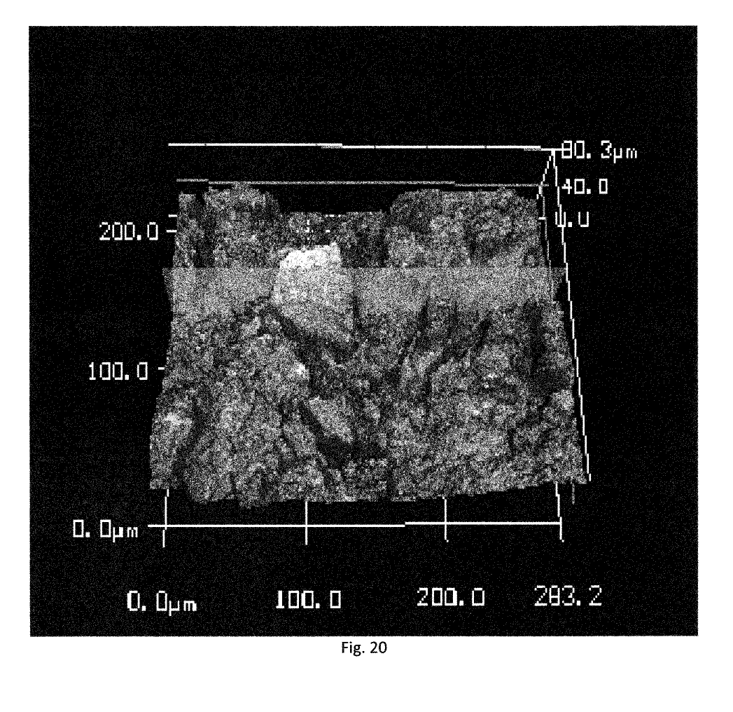

The surface of iron coated AZ31 magnesium alloy with an intermediate layer was evaluated using a VK-X100/X200. This surface roughness values give an idea about texture of the iron coated sample. The image of the sample was taken at 10× magnification. FIG. 17 shows the area taken for roughness evaluation. The average surface roughness was 10.4776 μm. This VK-X100/X200 gives the 3D image of the surface and shows the varying thickness on the surface due to surface dents. FIG. 18-21 show images of the roughness evaluation of the surface.

### Thickness Evaluation

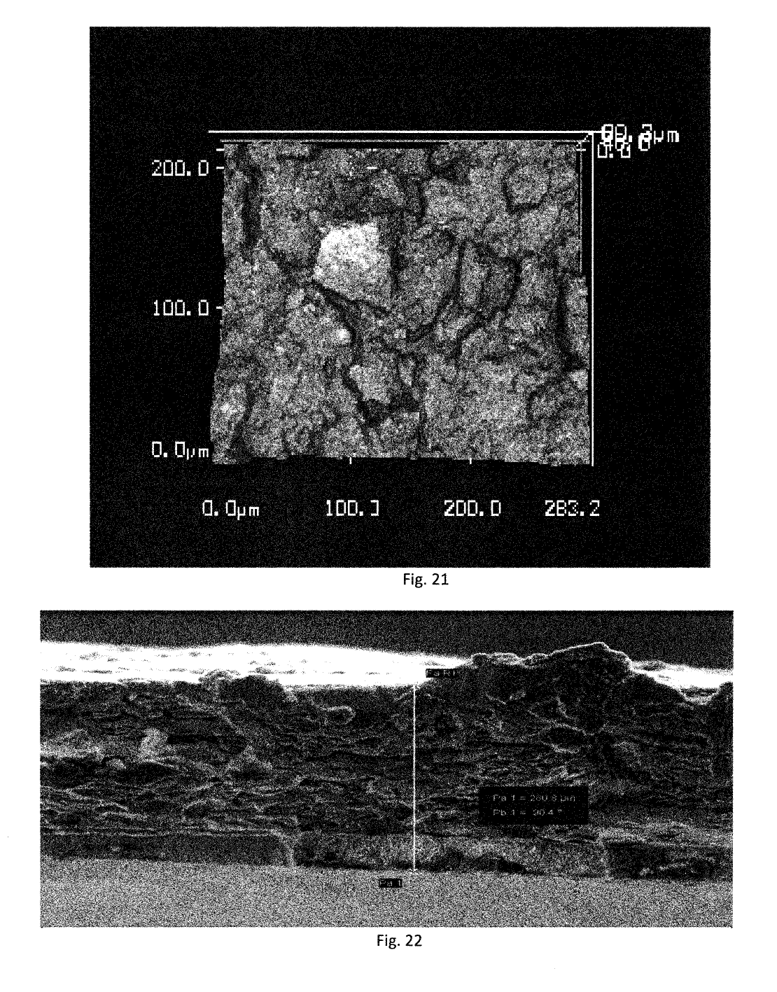

The side view of the broken sample was taken using an SEM. The sample was broken up after dipping it in liquid nitrogen so as to get a clean break and not shear it. The entire sample thickness as measured by the SEM was 280.8 μm. The image in FIG. 22 was taken at a magnification of 100 μm. The thickness of the uncoated sample was 250 μm and the thickness measured after coating it was 280.8 μm. Thus, the final thickness of the iron layer was found to be 30.4 μm.

### Corrosion Evaluation

Accelerated corrosion tests were conducted on the iron coated AZ31 sample and compared with the uncoated AZ31 magnesium sample. Open circuit potential test and potentiodynamic polarization test were conducted on the coated and uncoated samples.

### Open Circuit Potential

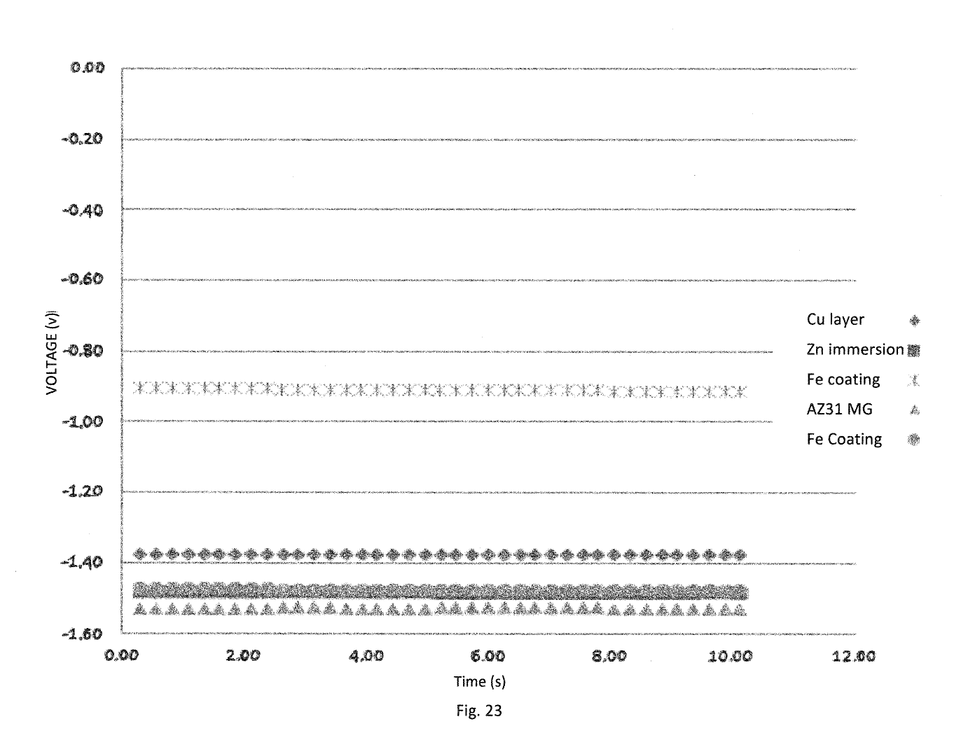

The open circuit potential (OCP) is also called as the corrosion potential or equilibrium potential is the potential at which there is no current. OCP of different layers and uncoated samples were evaluated using a potentiostat/galvenostat in PBS solution over a period of 10 seconds. The results were overlaid using excel. The values for the data entered are shown in methodology. Higher OCP refers to higher corrosion resistance. FIG. 23 shows the OCP comparison. Iron coated AZ31 without an intermediate copper showed very low OCP of −1.5V which was same as zinc immersion coated AZ31 substrate. OCP of copper coated AZ31 was −1.42V and OCP of iron coated AZ31 magnesium with an intermediate copper layer was −0.90V. There was an increasing trend in OCP values as the number of layers increased. The OCP curve justifies the presence of an intermediate copper layer for electroplating of iron on AZ31 magnesium alloy.

### Potentiodynamic Polarization

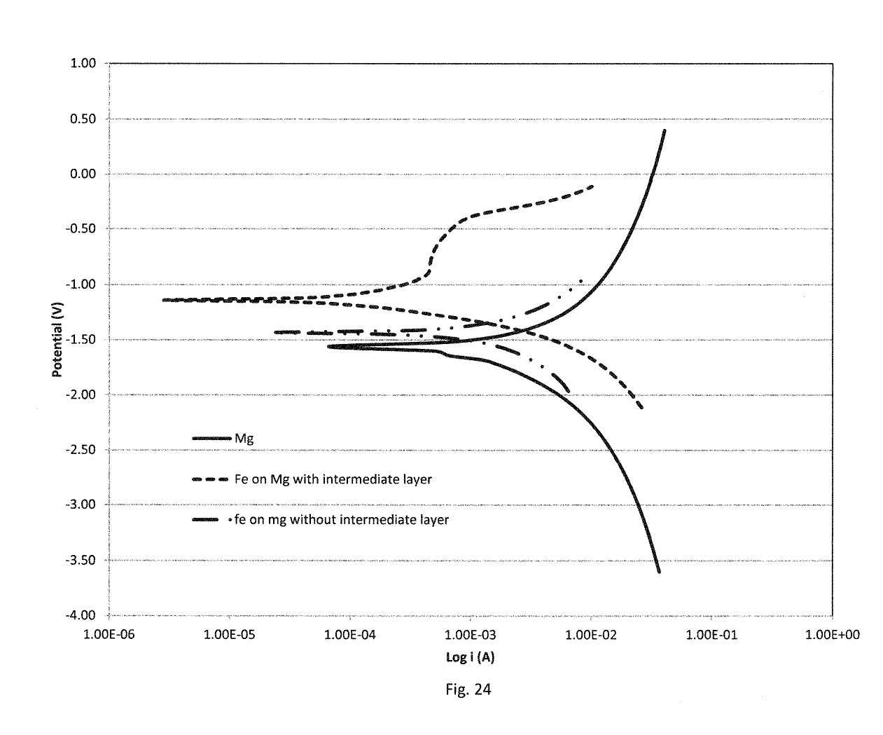

The tafel technique (FIG. 24) can be used to get an accurate value of corrosion rate of a metal in a solution. The corrosion current is measured during a sweep of potential. The voltage was swept from −250 mV to 250 mV relative to Eoc. The data is fitted against a standard model (butler-volmer model) which gives an estimate of Icon which helps to calculate a corrosion rate (Burstein, 2005). The graphical output of the experiment is a plot of log current versus potential. From the tafel curve it is possible to obtain corrosion current, corrosion rate, kinetic information for the anodic and cathodic reactions. The tafel test was run using a GAMRY reference 600™ potentiostat/galvenostat with the iron coated AZ31 magnesium sample in PBS solution. The results were overlaid with the results of uncoated AZ31 magnesium samples and coated magnesium without an intermediate layer.

TABLE
Comparison of corrosion rates of Iron coated AZ31 with a copper
layer, Iron coated AZ31 without a copper layer and pure AZ31

| | Iron Coated | | Iron coated | | | |
|---|---|---|---|---|---|---|
| | AZ31 with a | | AZ31 without a | | | |
| | copper layer | | copper layer | | Pure AZ31 | |
| Beta A | 482.1e⁻³ | V/decade | 1.043 | V/decade | 1.281 | V/decade |
| Beta C | 241.6e⁻³ | V/decade | 1.641 | V/decade | 1.793 | V/decade |
| Icorr | 147.0 | μA | 3.800 | mA | 4.300 | Ma |
| Ecorr | −1.140 | V | −1.440 | V | −1.570 | V |

| | | | |
|---|---|---|---|
| Corrosion | 54.08 mpy | 1.089e³ mpy | 1.591e³ mpy |
| Rate | (1.373632 mm/yr) | (27.6606 mm/yr) | (40.4114 mm/yr) |

After the plots were overlaid it was found that the corrosion rate of iron coated AZ31 magnesium with an intermediate copper layer offered better corrosion resistance than the uncoated AZ31 magnesium and iron coated AZ31 magnesium without an intermediate layer. More the graph is to the right the corrosion current increases which means an increase in the rate of corrosion. Iron coated AZ31 magnesium with an intermediate was found higher and to the left of the other graphs which says that the corrosion current decreases and the corrosion voltage increases. Higher open circuit means higher corrosion resistance. The FIG. 24 shows the tafel plots of iron coated AZ31 magnesium with an intermediate, iron coated AZ31 magnesium without an intermediate layer and uncoated AZ31 magnesium alloy.

### Conclusion

In this work electroplating of iron on AZ31 magnesium alloy has been demonstrated. A protocol for electroplating iron on magnesium was developed. Presence of iron was proved by EDX analysis. Iron can be plated only at low pH, and since magnesium corrodes at a low pH, the surface of magnesium had to be made non-reactive. An intermediate copper layer was electroplated on the surface of magnesium. This intermediate layer prevented attack by the acidic electrolyte. A zinc immersion layer was formed before the copper layer to the prevent magnesium interaction with copper electrolyte and also to ensure better adhesion of copper on the magnesium surface. OCP curves (FIG. 4.21) show the increase of potential during every stage. The potential is the lowest for bare magnesium and it was the highest for iron coated magnesium with an intermediate copper layer. Presence of iron was confirmed by EDX analysis which gives the elemental composition.

Thickness of the coating was found to be 21.8μm and surface roughness was found to be 10.4476 μm. Corrosion rate from tafel plots shows the corrosion rate of iron coated magnesium is 1.373632 mm/yr and bare magnesium substrate is 40.4114 mm/yr, which is lower than bare magnesium substrate. All the corrosion tests were conducted in PBS.

This work mainly focuses on electroplating iron on AZ31 magnesium alloy so that it can be used as biodegradable arterial stents and offer better corrosion resistance than the bare Magnesium substrate. Additional work will be aimed at coating iron directly. Using ferric chloride salt instead of ferrous ammonium sulfate might help in running the process at better current density and increase the throwing power of the cathode.

The current technology of stents is drug eluting stents which release drugs constantly over a period of time. The iron coated stents can be made to function as drug eluting stents by a Self Assembled Monolayer (SAM) coating. The drug molecules can be attached on to the tail of the SAM's.

### Example 2

### Deposition of Niobium on Magnesium

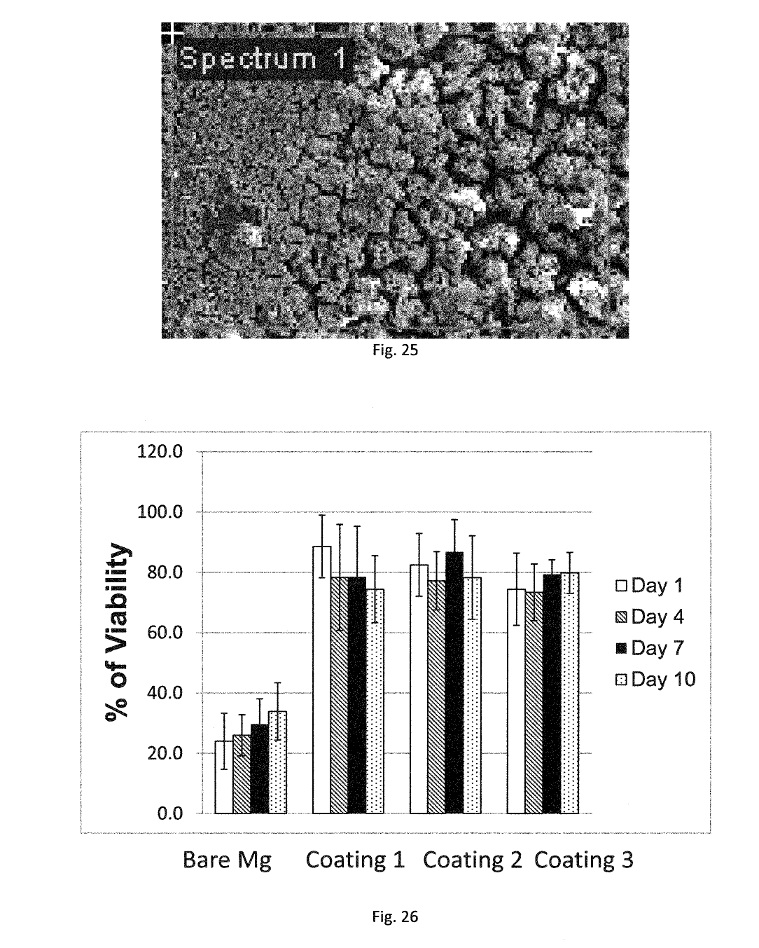

In this example, a non-biodegradable metal was deposited directly onto a magnesium-based substrate. FIG. 25 shows the deposited coating. The deposition of niobium was confirmed using EDS. Ultrasonication of the niobium deposited samples was carried out to remove any loosely bound niobium from the surface. The EDS spectrum analysis results show that before post niobium deposition (before ultrasonication), there was a 38% wt deposition of niobium. After ultrasonication there was a 35.5% wt deposition of niobium confirming formation of electrodeposition of niobium on magnesium.

### Example 3

### Deposition of an Organic Coating on Magnesium

An organic coating was deposited onto the magnesium substrate. Deposition of the organic (polymeric layers) layers was carried out using deposition and dip coating techniques. The biocompatibility as compared to bare magnesium is shown in FIG. 26. The coatings chosen were polyester based organic layers. Coating 1 and coating 2 differed in molecular weight of the polymers while coating 3 was an alternative polymer within the polyester class. This illustrates that enhancement of functionality (biocompatibility) of magnesium through use of these coatings.

What is claimed:

1. An implantable device comprising:
a body having an external surface, said body formed at least in part by metallic magnesium or an alloy thereof; and
a biocorrosion-inhibiting film on at least a portion of said external surface, wherein said biocorrosion-inhibiting film comprises a metal.

2. The implantable device of claim 1, wherein said body is selected from the group consisting of: screws, plates, nails, pins, rods, hooks, staples, wires, clamps, clips, stents, anchors, porous bone scaffolds, porous cartilage scaffolds, vertebral body replacements, dental implants, and combinations thereof.

3. The implantable device of claim 1, wherein said bioconosion-inhibiting film consists essentially of metal.

4. The implantable device of claim 1, wherein said metal is biodegradable.

5. The implantable device of claim 1, wherein said metal is selected from the group consisting of iron, niobium, titanium, tantalum, zinc, copper, cobalt, chromium, nitinol, combinations thereof, and alloys thereof.

6. The implantable device of claim 1, wherein said biocorrosion-inhibiting film is electroplated onto said external surface.

7. The implantable device of claim 1, wherein said biocorrosion-inhibiting film is porous.

8. The implantable device of claim 1, further comprising:
an intermediate film between said external surface and said biocorrosion-inhibiting film.

9. The implantable device of claim 8, wherein said intermediate film comprises a metal.

10. The implantable device of claim 8, wherein said intermediate film is non-porous.

11. The implantable device of claim 8, further comprising an adhesion promoting film between said external surface and said intermediate film.

12. The implantable device of claim 11, wherein said adhesion promoting film is non-porous.

13. The implantable device of claim 11, wherein said adhesion promoting film comprises a metal.

14. The implantable device of claim 1, further comprising a functional coating on said biocorrosion-inhibiting film.

15. The implantable device of claim 14, wherein said functional coating comprises one or more active agents selected from the group consisting of therapeutic agents, antioxidants, anesthetic agents, anti-proliferative agents, osteoinductive agents, osteoconductive agents, and combinations thereof.

16. The implantable device of claim 15, wherein said active agent is dispersed in a cattier or matrix.

17. A method of inhibiting corrosion on a magnesium-based implantable device, said method comprising:
providing a body having an external surface, said body formed at least in part by metallic magnesium or an alloy thereof; and
depositing a biocorrosion-inhibiting film on at least a portion of said external surface, wherein said biocorrosion-inhibiting film comprises a metal.

18. The method of claim 17, wherein said depositing is selected from the group consisting of electroplating, sputtering and vapor deposition.

19. The method of claim 17, wherein said biocorrosion inhibiting film is selectively deposited onto discrete areas of said external surface.

20. The method of claim 17, further comprising, before depositing said biocorrosion-inhibiting film:
depositing an optional adhesion promoting film onto said external surface; and
depositing an intermediate film onto said adhesion promoting film, wherein said biocorrosion-inhibiting film is deposited onto said intermediate film.
